(19) United States
(12) Patent Application Publication
(10) Pub. No.: US 2005/0239759 A1
(43) Pub. Date: Oct. 27, 2005

(54) Method of treatment of disease using an adenosine A1 receptor antagonist and an aldosterone inhibitor

(76) Inventors: Lauren Otsuki, San Diego, CA (US); Kenneth Widder, Rancho Santa Fe, CA (US); Howard C. Dittrich, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/107,637

(22) Filed: Apr. 14, 2005

Related U.S. Application Data
(60) Provisional application No. 60/563,166, filed on Apr. 16, 2004.

(51) Int. Cl. A61K 31/56; A61K 31/4745

(52) U.S. Cl. 514/171; 514/303

(57) ABSTRACT

Pharmaceutical compositions comprising an aldosterone inhibitor and an adenosine A₁ receptor antagonist (AA₁RA) and methods of treating cardiovascular disease comprising identifying a patient in need of such treatment, and administering a pharmaceutical composition disclosed herein to said patient are disclosed.

### RELATED APPLICATION DATA

The present application claims priority to U.S. Provisional Application Ser. No. 60/563,166, filed on Apr. 16, 2004, by Otsuki et al., and entitled METHOD OF TREATMENT OF DISEASE USING AN ADENOSINE A1 RECEPTER ANTAGONIST AND AN ALDOSTERONE INHIBITOR, which is incorporated by reference herein in its entirety.

### FIELD OF THE INVENTION

The present invention relates to compositions comprising a combination of an adenosine A1 receptor antagonist and an aldosterone inhibitor and methods of treatment of patients suffering from cardiac disease with said compositions.

### SUMMARY OF THE INVENTION

Disclosed is a pharmaceutical composition comprising an aldosterone inhibitor and an adenosine A₁ receptor antagonist (AA₁RA).

Also disclosed is a method of treating cardiovascular disease comprising identifying a patient in need of such treatment, and administering a pharmaceutical composition disclosed herein to said patient.

### DETAILED DESCRIPTION OF THE PREFERRED EMBODIMENT

Aspects of the present invention relate to the treatment of cardiovascular diseases using a combination of an aldosterone inhibitor and an adenosine A₁ receptor antagonists, or AA₁RA. Each of these compounds have individually been shown to be somewhat effective in the treatment of cardiovascular disease, such as congestive heart failure, hypertension, asymptomatic left ventricular dysfunction, or inflammation of the vasculature, such as coronary artery disease.

A number of aldosterone inhibitors are commercially available. These compounds include, but are not limited to, spironolactone (ALDACTONE®) and eplerenone (INSPRA®). The scope of the present invention includes all those aldosterone inhibitors now known and all those aldosterone inhibitors to be discovered in the future.

A number of AA₁RAs are known in the art, though currently, none are commercially available as a therapeutic. AA₁RAs antagonize the A₁ receptor of adenosine selectively. The majority of the known AA₁RAs are derivatives of xanthine and include compounds such as 1,3-dipropyl-8-{3-oxatricyclo[3.1.2.0.^{2,4}]oct-6(7)-yl}xanthine (also known as 1,3-dipropyl-8-[5,6-exo-epoxy-2(S)norbornyl]xanthine, ENX, CVT-124, and BG9719), 8-(3-noradamantyl)-1,3-dipropylxanthine (also known as KW-3902), theophyllilne, and caffeine. Other AA₁RAs are disclosed in U.S. Pat. Nos. 5,446,046, 5,631,260, and 5,668,139, the specification of all of which is hereby incorporated by reference herein in their entirety, including any drawings. The scope of the present invention includes all those AA₁RAs now known and all those AA₁RAs to be discovered in the future.

A significant problem encountered in treating certain conditions with individual medications is that following a course of therapy the patients become refractory to the treatment, i.e., the patients begin to respond less and less to the medication until they do not respond at all. This problem is very common in patients who suffer from, for example, congestive heart failure, and are treated with diuretics.

Individual diuretics act on a specific segment of nephrons, e.g., proximal tubule, loop of Henle, or distal tubule. One mechanism by which diuretics increase urine volume is that they inhibit reabsorption of sodium and accompanying water passing through the nephron. Thus, for example, a loop diuretic inhibits reabsorption in the loop of Henle. As a consequence, higher concentrations of sodium are passed downstream to the distal tubule. This initially results in a greater volume of urine, hence the diuretic effect. However, the distal portion of the tubule recognizes the increase in sodium concentration and the kidney reacts in two ways; one is to increase sodium reabsorption elsewhere in the nephron; the other is to feedback via adenosine A₁ receptors to the afferent arteriole where vasoconstriction occurs. This feedback mechanism is known as tubuloglomerular feedback (TGF). This vasoconstriction results in decreased renal blood flow and decreased glomerular filtration rate (GFR). With time, these two mechanisms result in a decrease in diuretic effect and worsening of renal function. This sequence of events contributes to the progression of disease.

AA₁RAs act on the afferent arteriole of the kidney to produce vasodilation and thereby improve renal blood flow in patients with CHF. They also block the TGF mechanism mediated by adenosine (via A₁ receptors) described above. This ultimately allows for increased GFR and improved renal function. In addition, AA₁RAs inhibit the reabsorption of sodium (and, therefore, water) in the proximal tubule, which results in diuresis.

AA₁RAs exert a diuretic effect by inhibiting the reabsorption of sodium in the proximal tubule of the nephron through adenosine Al receptors, In addition, AA₁RAs improve renal blood flow and glomerular filtration by inhibiting TGF, which is activated by diuretics that increase distal tubular sodium. Further, it appears that AA₁RAs have antioxidant properties in some conditions, such as radiographic contrast-mediated nephropathy, and therefore, may have similar properties in other conditions where oxygen-free radicals are injurious.

Aldosterone inhibitors block aldosterone binding at the mineralocorticoid receptors. These compounds prevent the induction of sodium reabsorption in kidney, heart, blood vessels, and brain that can lead to harmful effects, such as increased blood pressure. It is also known that aldosterone inhibitors may inhibit vascular inflammation that is mediated by aldosterone.

The combination of the invention described herein acts synergistically to further improve the condition of patients with hypertension or CHF. The diuretic effect of AA₁RAs, specially in salt-sensitive hypertensive patients along with the inhibition of aldosterone decreases blood pressure through two different mechanisms, whose effects build on one another. In addition, most CHF patients are also on additional diuretics. The combination allows for greater efficacy of other more distally acting diuretics by improving renal blood flow and renal function.

Furthermore, the combinations of the present invention are superior since AA₁RAs will allow increased effectiveness of aldosterone inhibitors by increasing renal perfusion and delivery of the aldosterone inhibitor to its site of action in the kidney. Further, since AA₁RAs induce the renin-angiotensin-aldosterone system, the combined use of these two compounds is superior with regard to congestive heart failure, hypertension, myocardial infarction, or renal disease.

In addition, because of the tissue damage induced by aldosterone through endothelin and the creation of reactive oxygen species, there is a belief that any cardiovascular condition in which endothelial tissues suffer from inflammation (e.g., atherosclerosis, myocardial infarction, and the like) a benefit may be derived from aldosterone inhibitors. The combination of aldosterone inhibitors with AA₁RAs would further inhibit these oxidative processes and therefore prove beneficial in the prevention, and treatment, of such conditions.

Thus, in a first aspect, the invention relates to a pharmaceutical composition comprising an aldosterone inhibitor and an adenosine A₁ receptor antagonist (AA₁RA). The aldosterone inhibitor may be selected from the group consisting of spironolactone and eplerenone, or a pharmaceutically acceptable salt, prodrug, ester, or amide thereof. However, the inclusion of other aldosterone inhibitors is within the scope of the present invention.

The AA₁RA may be a xanthine-derivative compound of Formula I or a pharmaceutically acceptable salt thereof, 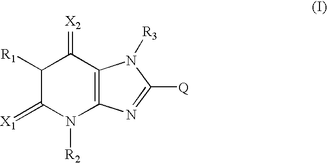
where
each of X₁ and X₂ independently represents oxygen or sulfur;
Q represents: 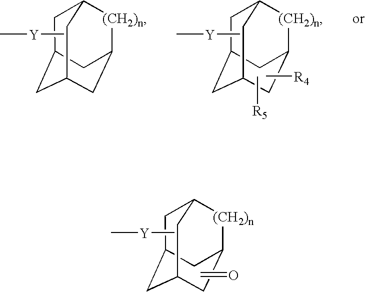
   where Y represents a single bond or alkylene having 1 to 4 carbon atoms, n represents 0 or 1;
each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or
R₄ and R₅ are the same or different and each represent hydrogen or hydroxy, and when both R₄ and R₅ are hydrogen, at least one of R₁ and R₂ is hydroxy-substituted or oxo-substituted lower alkyl,
   provided that when Q is 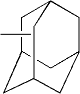
   then R₁, R₂ and R₃ are not simultaneously methyl.

In some embodiments, both of R₁ and R₂ of the compound of Formula I are lower alkyl and R₃ is hydrogen; and both of X₁ and X₂ are oxygen. In other embodiments, R₁, R₂ and R₃ independently represents hydrogen or lower alkyl. In still other embodiments, each of R₁ and R₂ independently represents allyl or propargyl and R₃ represents hydrogen or lower alkyl. In certain embodiments, X₁ and X₂ are both oxygen and n is 0.

In some embodiments, R₁ is hydroxy-substituted, oxo-substituted or unsubstituted propyl; R₂ is hydroxy-substituted or unsubstituted propyl; and Y is a single bond. In other embodiments, R₁ is propyl, 2-hydroxypropyl,2-oxopropyl or 3-oxopropyl; R₂ is propyl, 2-hydroxypropyl or 3-hydroxypropyl.

In some embodiments Q is 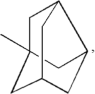
while in other embodiments Q is

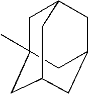

In other embodiments, Q is 9-hydroxy, 9-oxo or 6-hydroxy substituted 3tricyclo[3.3.1.0^{3,7}]nonyl, or 3-hydroxy-1tricyclo[3.3.1.1^{3,7}]decyl.

In certain embodiments, the AA₁RA is selected from the group consisting of 8-(noradamantan-3-yl)-1,3-dipropylxanthine; 1,3-Diallyl-8-(3-noradamantyl)xanthine, 3-allyl-8-(3-noradamantyl)1-propargylxanthine, tricyclo[3.3.1.0^{3,7}]nonyl)-1,3-dipropylxanthine (also referred to as “M1-trans”), 8-(cis-9-hydroxy-3-tricyclo[3.3.1.0^{3,7}]nonyl)-1,3-dipropylxanthine (also referred to as “M1-cis”), 8-(trans-9-hydroxy-3-tricyclo[3.3.1.0^{3,7}]nonyl)-1-(2-oxopropyl)-3-propylxanthine and 1-)2-hydroxypropyl)-8-(trans-9-hydroxy-3-tricyclo[3.3.1.0^{3,7}]nonyl)-3-propylxanthine, or a pharmaceutically acceptable salt thereof.

In other embodiments, the AA₁RA is a xanthine epoxide-derivative compound of Formula II or Formula III, or a pharmaceutically acceptable salt thereof, 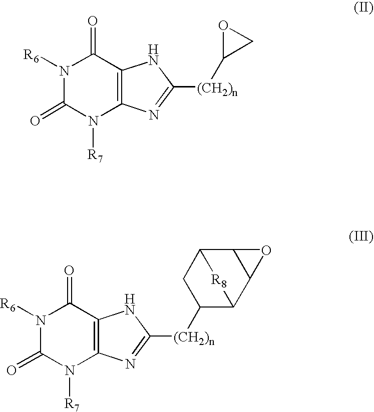
where R₆ and R₇ are the same or different, and can be hydrogen or an alkyl group of 1-4 carbons, R₈ is either oxygen or (CH₂)₁₋₄, and n=0-4.

The xanthine epoxide-derivative compound may be

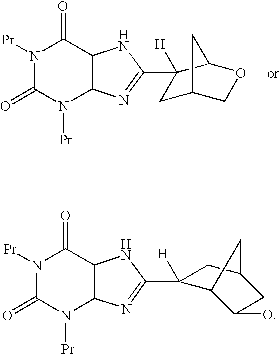

In another aspect, the invention relates to a method of treating cardiovascular disease or renal disease comprising identifying a patient in need of such treatment, and administering a pharmaceutical composition as described herein to said patient. In certain embodiments, the patient may be a mammal. The mammal may be selected from the group consisting of mice, rats, rabbits, guinea pigs, dogs, cats, sheep, goats, cows, primates, such as monkeys, chimpanzees, and apes, and humans. In some embodiments, the patient is a human.

In some embodiments, the administering step comprises administering said aldosterone inhibitor and said AA₁RA nearly simultaneously. These embodiments include those in which the AA₁RA and the aldosterone inhibitor are in the same administrable composition, i.e., a single tablet, pill, or capsule, or a single solution for intravenous injection, or a single drinkable solution, or a single dragee formulation or patch, contains both compounds. The embodiments also include those in which each compound is in a separate administrable composition, but the patient is directed to take the separate compositions nearly simultaneously, i.e., one pill is taken right after the other or that one injection of one compound is made right after the injection of another compound, etc.

In other embodiments the administering step comprises administering one of the aldosterone inhibitor and the AA₁RA first and then administering the other one of the aldosterone inhibitor and the AA₁RA. In these embodiments, the patient may be administered a composition comprising one of the compounds and then at some time, a few minutes or a few hours, later be administered another composition comprising the other one of the compounds. Also included in these embodiments are those in which the patient is administered a composition comprising one of the compounds on a routine or continuous basis while receiving a composition comprising the other compound occasionally.

The methods of the present invention are intended to provide treatment for cardiovascular disease, which may include congestive heart failure, hypertension, asymptomatic left ventricular dysfunction, coronary artery disease, or acute myocardial infarction. In some instances, patients suffering from a cardiovascular disease are in need of after-load reduction. The methods of the present invention are suitable to provide treatment for these patients as well.

In another aspect, the invention relates to a pharmaceutical composition comprising a combination of an AA₁RA and an aldosterone inhibitor, as described above, and a physiologically acceptable carrier, diluent, or excipient, or a combination thereof.

The term “pharmaceutical composition” refers to a mixture of a compound of the invention with other chemical components, such as diluents or carriers. The pharmaceutical composition facilitates administration of the compound to an organism. Multiple techniques of administering a compound exist in the art including, but not limited to, oral, injection, aerosol, parenteral, and topical administration. Pharmaceutical compositions can also be obtained by reacting compounds with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid and the like.

The term “carrier” defines a chemical compound that facilitates the incorporation of a compound into cells or tissues. For example dimethyl sulfoxide (DMSO) is a commonly utilized carrier as it facilitates the uptake of many organic compounds into the cells or tissues of an organism.

The term “diluent” defines chemical compounds diluted in water that will dissolve the compound of interest as well as stabilize the biologically active form of the compound. Salts dissolved in buffered solutions are utilized as diluents in the art. One commonly used buffered solution is phosphate buffered saline because it mimics the salt conditions of human blood. Since buffer salts can control the pH of a solution at low concentrations, a buffered diluent rarely modifies the biological activity of a compound.

The term “physiologically acceptable” defines a carrier or diluent that does not abrogate the biological activity and properties of the compound.

The pharmaceutical compositions described herein can be administered to a human patient per se, or in pharmaceutical compositions where they are mixed with other active ingredients, as in combination therapy, or suitable carriers or excipient(s). Techniques for formulation and administration of the compounds of the instant application may be found in “Remington's Pharmaceutical Sciences,” Mack Publishing Co., Easton, Pa., 18th edition, 1990.

Suitable routes of administration may, for example, include oral, rectal, transmucosal, or intestinal administration; parenteral delivery, including intramuscular, subcutaneous, intravenous, intramedullary injections, as well as intrathecal, direct intraventricular, intraperitoneal, intranasal, or intraocular injections.

Alternately, one may administer the compound in a local rather than systemic manner, for example, via injection of the compound directly in the renal or cardiac area, often in a depot or sustained release formulation. Furthermore, one may administer the drug in a targeted drug delivery system, for example, in a liposome coated with a tissue-specific antibody. The liposomes will be targeted to and taken up selectively by the organ.

The pharmaceutical compositions of the present invention may be manufactured in a manner that is itself known, e.g., by means of conventional mixing, dissolving, granulating, dragee-making, levigating, emulsifying, encapsulating, entrapping or tabeleting processes.

Pharmaceutical compositions for use in accordance with the present invention thus may be formulated in conventional manner using one or more physiologically acceptable carriers comprising excipients and auxiliaries which facilitate processing of the active compounds into preparations which can be used pharmaceutically. Proper formulation is dependent upon the route of administration chosen. Any of the well-known techniques, carriers, and excipients may be used as suitable and as understood in the art; e.g., in Remington's Pharmaceutical Sciences, above.

For injection, the agents of the invention may be formulated in aqueous solutions or lipid emulsions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained by mixing one or more solid excipient with pharmaceutical combination of the invention, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethyl cellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacqucr solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Furthermore, the formulations of the present invention may be coated with enteric polymers. All formulations for oral administration should be in dosages suitable for such administration.

For buccal administration, the compositions may take the form of tablets or lozenges formulated in conventional manner.

For administration by inhalation, the compounds for use according to the present invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, e.g., sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase. A common cosolvent system used is the VPD co-solvent system, which is a solution of 3% w/v benzyl alcohol, 8% w/v of the nonpolar surfactant Polysorbate 80™, and 65% w/v polyethylene glycol 300, made up to volume in absolute ethanol. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of POLYSORBATE 80™; the fraction size of polyethylene glycol may be varied; other biocompatible polymers may replace polyethylene glycol, e.g., polyvinyl pyrrolidone; and other sugars or polysaccharides may substitute for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

Some emulsions used in solubilizing and delivering the xanthine derivatives described above are discussed in U.S. Pat. No. 6,210,687, which is incorporated by reference herein in its entirety, including any drawings.

Many of the compounds used in the pharmaceutical combinations of the invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents than are the corresponding free acid or base forms.

Pharmaceutical compositions suitable for use in the present invention include compositions where the active ingredients are contained in an amount effective to achieve its intended purpose. More specifically, a therapeutically effective amount means an amount of compound effective to prevent, alleviate or ameliorate symptoms of disease or prolong the survival of the subject being treated. Determination of a therapeutically effective amount is well within the capability of those skilled in the art, especially in light of the detailed disclosure provided herein.

The exact formulation, route of administration and dosage for the pharmaceutical compositions of the present invention can be chosen by the individual physician in view of the patient's condition. (See e.g., Fingl et al. 1975, in “The Pharmacological Basis of Therapeutics”, Ch. 1 p. 1). Typically, the dose range of the composition administered to the patient can be from about 0.5 to 1000 mg/kg of the patient's body weight. The dosage may be a single one or a series of two or more given in the course of one or more days, as is needed by the patient.

The daily dosage regimen for an adult human patient may be, for example, an oral dose of between 0.1 mg and 500 mg, preferably between 1 mg and 250 mg, e.g. 5 to 200 mg or an intravenous, subcutaneous, or intramuscular dose of between 0.01 mg and 100 mg, preferably between 0.1 mg and 60 mg, e.g. 1 to 40 mg of the pharmaceutical compositions of the present invention or a pharmaceutically acceptable salt thereof calculated as the free base, the composition being administered 1 to 4 times per day. Alternatively the compositions of the invention may be administered by continuous intravenous infusion, preferably at a dose of up to 400 mg per day. Thus, the total daily dosage by oral administration will be in the range 1 to 2000 mg and the total daily dosage by parenteral administration will be in the range 0.1 to 400 mg. Suitably the compounds will be administered for a period of continuous therapy, for example for a week or more, or for months or years.

Dosage amount and interval may be adjusted individually to provide plasma levels of the active moiety which are sufficient to maintain the modulating effects, or minimal effective concentration (MEC). The MEC will vary for each compound but can be estimated from in vitro data. Dosages necessary to achieve the MEC will depend on individual characteristics and route of administration. However, HPLC assays or bioassays can be used to determine plasma concentrations.

Dosage intervals can also be determined using MEC value. Compositions should be administered using a regimen which maintains plasma levels above the MEC for 10-90% of the time, preferably between 30-90% and most preferably between 50-90%.

In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Compositions comprising a compound of the invention formulated in a compatible pharmaceutical carrier may also be prepared, placed in an appropriate container, and labeled for treatment of an indicated condition.

1. A pharmaceutical composition comprising an aldosterone inhibitor and an adenosine A₁ receptor antagonist (AA₁RA).

2. The composition of claim 1, wherein said aldosterone inhibitor is selected from the group consisting of spironolactone and eplerenone, or a pharmaceutically acceptable salt, prodrug, ester, or amide thereof.

3. The composition of claim 1, wherein said AA₁RA is a xanthine-derivative compound of Formula I or a pharmaceutically acceptable salt thereof, 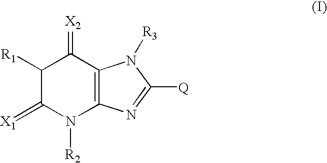
wherein
each of X₁ and X₂ independently represents oxygen or sulfur;
Q represents: 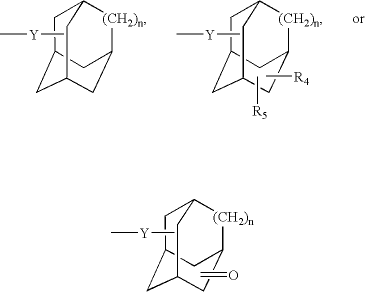
where Y represents a single bond or alkylene having 1 to 4 carbon atoms, n represents 0 or 1;
each of R₁ and R₂ independently represents hydrogen, lower alkyl, allyl, propargyl, or hydroxy-substituted, oxo-substituted or unsubstituted lower alkyl, and R₃ represents hydrogen or lower alkyl, or
R₄ and R₅ are the same or different and each represent hydrogen or hydroxy, and when both R₄ and R₅ are hydrogen, at least one of R₁ and R₂ is hydroxy-substituted or oxo-substituted lower alkyl,
provided that when Q is 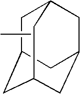
then R₁, R₂ and R₃ are not simultaneously methyl.

4. The composition of claim 3, wherein both of R₁ and R₂ are lower alkyl and R₃ is hydrogen; and both of X₁ and X₂ are oxygen.

5. The composition of claim 3, wherein each of R₁, R₂ and R₃ independently represents hydrogen or lower alkyl.

6. The composition of claim 3, wherein each of R₁ and R₂ independently represents allyl or propargyl and R₃ represents hydrogen or lower alkyl.

7. The composition of claim 3, wherein R₁ is hydroxy-substituted, oxo-substituted or unsubstituted propyl; R₂ is hydroxy-substituted or unsubstituted propyl; and Y is a single bond.

8. The composition of claim 3, wherein R₁ is propyl, 2-hydroxypropyl, 2-oxopropyl or 3-oxopropyl; R₂ is propyl, 2-hydroxypropyl or 3-hydroxypropyl.

9. The composition of claim 6, wherein X₁ and X₂ are both oxygen and n is 0.

10. The composition of claim 5, wherein Q is

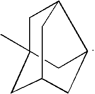

11. The composition of claim 5, wherein Q is

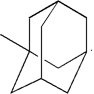

12. The composition of claim 5, wherein Q is 9-hydroxy, 9-oxo or 6-hydroxy substituted 3tricyclo[3.3.1.0^{3,7}]nonyl, or 3-hydroxy-1tricyclo[3.3.1.1^{3,7}]decyl.

13. The composition of claim 1, wherein said AA₁RA is selected from the group consisting of 8-(noradamantan-3-yl)-1,3-dipropylxanthine; 1,3-Diallyl-8-(3-noradamantyl)xanthine, 3-allyl-8-(3-noradamantyl)-1-propargylxanthine, 8-(trans-9-hydroxy-3-tricyclo[3.3.1.0^{3,7}]nonyl)-1,3-dipropylxanthine, 8-(cis-9-hydroxy-3-tricyclo[3.3.1.0^{3,7}]nonyl)-1,3-dipropylxanthine, 8-(trans-9-hydroxy-3-tricyclo[3.3.1.0^{3,7}]nonyl)-1-(2-oxopropyl)-3-propylxanthine and 1-(2-hydroxypropyl)-8(trans-9-hydroxy-3-tricyclo[3.3.1.0^{3,7}]nonyl)-3-propylxanthine, or a pharmaceutically acceptable salt thereof.

14. The composition of claim 1, wherein said AA₁RA is a xanthine epoxide-derivative compound of Formula II or Formula III, or a pharmaceutically acceptable salt thereof, 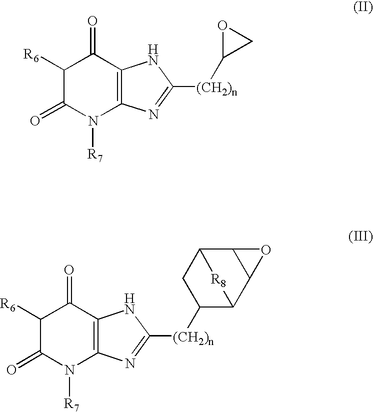
wherein R₆ and R₇ are the same or different, and can be hydrogen or an alkyl group of 1-4 carbons, R₈ is either oxygen or (CH₂)₁₄, and n=0-4.

15. The composition of claim 1, wherein said xanthine epoxide-derivative compound is

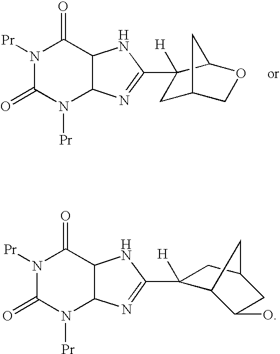

16. A method of treating cardiovascular disease comprising identifying a patient in need of such treatment, and administering a pharmaceutical composition of claim 1 to said patient.

17. The method of claim 16, wherein said administering step comprises administering said aldosterone inhibitor and said AA₁RA nearly simultaneously.

18. The method of claim 16, wherein said administering step comprises administering one of said aldosterone inhibitor and said AA₁RA first and then administering the other one of said aldosterone inhibitor and said AA₁RA.

19. The method of claim 16, wherein said cardiovascular disease is congestive heart failure, hypertension, asymptomatic left ventricular dysfunction, or coronary artery disease.

20. The method of claim 16, wherein said patient is in need of after-load reduction.

21. The method of claim 16, wherein said patient requires additional diuretic therapy or is refractory to diuretic therapy.
